(19) United States
(12) Patent Application Publication
(10) Pub. No.: US 2016/0076057 A1
(43) Pub. Date: Mar. 17, 2016

(54) INHIBITION OF METHANE AND HYDROGEN SULFIDE PRODUCTION IN ANAEROBIC DIGESTER ANIMAL FARMS, LANDFILLS, SEDIMENTS AND SEWER SYSTEMS

(71) Applicant: Innovative Environmental Technologies, Inc., Pipersville, PA (US)

(72) Inventors: Michael Scalzi, Doylestown, PA (US); Antonis Karachalios, North Wales, PA (US)

(21) Appl. No.: 14/488,635

(22) Filed: Sep. 17, 2014

(51) Int. Cl. C12P 5/02

(52) U.S. Cl. CPC C12P 5/023

(57) ABSTRACT

A method for inhibiting methane and hydrogen sulfide production from anaerobic digester systems and other biogas generating mediums disclosed. The biogas generating medium is contacted with an effective amount of a composition comprising red yeast rice and iron oxide to cause inhibition of methane and hydrogen sulfide production, and is useful in biogas generating medium from animal farms, including a swine, cattle or chicken farms. The method is useful to inhibit methane and hydrogen sulfide production in sewage systems, landfills, and sediment containing organic carbon. The disclosed inhibiting composition blocks 3-hydroxy-3-ethylglutaryl coenzyme A (HMG-CoA) reductase, and 8-hydroxy-5-deazaflavin (coenzyme F₄₂₀) in the methane production pathway, due to the presence of lovastatin in the red yeast rice. Furthermore the disclosed inhibiting composition prevents hydrogen sulfide formation with a competing reaction resulting into iron sulfate via reaction with iron (II) having been formed within the reducing environment from iron (III) oxide.

### FIELD OF THE DISCLOSURE

The disclosed method relates to the combined use of ferric iron oxide and red yeast rice extract to inhibit the methane and hydrogen sulfide production in anaerobic digester systems such as animal (swine, chicken, cattle) farms, landfills, sediments and enclosed sewer structures. Iron oxide will mainly address the excess hydrogen sulfide production in those systems by transforming it into insoluble iron sulfide, while the red yeast extract will target different enzymes and coenzymes systems that are responsible for the production of methane. The inhibition of those two gases will significantly lower the content of the biogas emissions and prevent reoccurring accidents, such as explosions, that have been observed in the aforementioned systems where methane and hydrogen sulfide concentrations are highly elevated.

### BACKGROUND

Anaerobic digestion is a common technology in today's agriculture, municipal waste, and brewing industries. It uses bacteria to break down waste organic materials into methane and other gases, which also have the potential to produce electricity or heat. It is a complex process that occurs in three basic stages as the result of the activity of a variety of microorganisms. Initially, a group of microorganisms converts organic material to a form that a second group of organisms uses to form organic acids. Methane-producing (methanogenic) anaerobic bacteria use these acids and complete the decomposition process. Biogas produced in anaerobic digesters consists of methane (50%-80%), carbon dioxide (20%-50%), and trace levels of other gases such as hydrogen, carbon monoxide, nitrogen, oxygen, and hydrogen sulfide.

Biogenic methane production, or methanogenesis, is a microbial process carried out by a unique class of prokaryotes. Methanogenic bacteria are a very diverse group of bacteria, in terms of structure and molecular traits. Hydrogen sulfide is produced during the anaerobic process, especially when high concentrations of sulfate are present in the influent.

### Animal Farms

Industrial farms, also called factory farms or CAFOs (confined animal feeding operations), pollute the air in many ways, emitting foul odors, airborne particles, greenhouse gases, and numerous toxic chemicals. United States farms alone produce more than 400 different gases, in addition to dust and airborne particles known as endotoxins generated during the handling and disposal of manure.

The USDA estimates that more than 335 million tons of manure is produced annually on U.S. farms. Stored for long periods of time in giant tanks or lagoons, the animal waste decomposes and pollutes the air with hundreds of different gases. These storage facilities are often located next to animal confinement facilities, with the livestock and the people who work with them continually exposed to harmful gases. Manure in storage facilities is an example of a biogas generating medium.

Hydrogen sulfide, methane, ammonia, and carbon dioxide are the major hazardous gases produced by decomposing manure. The EPA estimates that methane emissions from manure increased by 26 percent in the United States between 1990 and 2004, due primarily to larger, more concentrated dairy cow and swine facilities.

The digestion of manure occurs in four basic stages: hydrolysis, acidogenesis, acetogenesis and methanogenesis. The final stage, methanogenesis, is the step that breaks down the intermediate compounds to produce methane. The gas released during the digestion process is captured and can be burned. This biogas contains 50 to 70 percent methane, 30 to 50 percent carbon dioxide, and trace gases, including hydrogen sulfide. Methane-producing bacteria are most active in two temperature ranges, 95 to 105 degrees F. and 130 to 135 degrees F.

While manure is the largest contributor to air pollution from factory farms, industrial animal feed also plays a role. In 2004, the EPA estimated that 20 percent of all man-made methane production resulted from livestock digestion, primarily cows, which on factory farms are kept alive with low-quality grain-based feed that their bodies were not designed to digest. This feed fattens animals cheaply but causes chronic indigestion that contributes to higher methane emissions.

Generally, gases and odors produced in close confinement animal facilities are the result of bacterial action on biodegradable parts of animal waste. Gases produced in greatest volume are methane, carbon dioxide, ammonia and hydrogen sulfide.

There is also a complicated group of volatile organic compounds that contribute to the odor. These substances include amines, mercaptans, alcohols, carbonyls and sulfides in trace amounts.

Methane, the gas produced in greatest volume during the decomposition process, results from organic acids being degraded. Carbon dioxide is the second most abundant gas produced as organic acids are degraded. Ammonia is released as amino acids in protein are broken down by bacteria. Hydrogen sulfide is also a part of the odor. Anaerobic reduction of sulfur-containing compounds, such as certain amino acids, result in formation of hydrogen sulfide.

Under normal conditions, there is little likelihood of noxious gas levels rising to critical concentrations in a well-designed confinement facility. However, there are circumstances in which gas levels can become critically high, even when the facility is properly designed.

Ventilation breakdown is most often the cause of critically high gas levels in confinement facilities. Agitating manure that has been stored in a pit for several months can release dangerous quantities of noxious gases, even if the ventilation system is operating properly. The dangers during agitation are release of the highly toxic gas hydrogen sulfide and release of carbon dioxide in quantities sufficient to deplete the oxygen supply.

Entering a manure storage pit can be potentially lethal for humans. Carbon dioxide and hydrogen sulfide are heavier than air and tend to collect at the manure surface. In pits equipped with a cover or manhole opening only, methane can accumulate, creating potentially explosive conditions.

FIG. 1 shows a diagram of cycle of manure uses, and methane production from manure. As shown in the diagram, urine and feces, together with bedding form manure. The manure is stored in a manure pit or a digester, and oxygen is depleted for a large portion of the manure in the manure pit. Methanogenic bacteria breaks down the manure into biogas, with approximately 70% being CH₄, and approximately 30% being CO₂. The biogas can be used to power electrical generators and add power to an electrical grid. The biogas will also be burned with a flare to burn excess gas and reduce greenhouse gas emissions.

Residuals from the methanogenic bacteria process include liquids, which are used to fertilize farm land, and biosolids, with ammonium concentrated P and K. The biosolids are used to make solid manure fertilizer, and to make bedding which is used for animal bedding, and which is added into manure.

### Landfills

Landfill gas is produced through bacterial decomposition, volatilization and chemical reactions. Most landfill gas is produced by bacterial decomposition that occurs when organic waste solids, food, garden waste, wood and paper products, are broken down by bacteria naturally present in the waste and in soils. The organic waste solids, together with bacteria, are one example of a biogas generating medium. Volatilization generates landfill gas when certain wastes change from a liquid or solid into a vapor. Chemical reactions occur when different waste materials are mixed together during disposal operations.

In general, during anaerobic conditions, the composition of landfill gas is approximately 50 percent methane and 50 percent carbon dioxide with trace amounts (<1 percent) of nitrogen, oxygen, hydrogen sulfide, hydrogen, and non-methane organic compounds (NMOCs).

Sulfides are naturally occurring gases that often give a landfill gas mixture its unpleasant odor even at very low concentrations. Hydrogen sulfide is a colorless, flammable gas and is one of the most common sulfides responsible for landfill odors.

Hydrogen sulfide is found usually by landfilling of solid waste. The concentration of hydrogen sulfide detected in landfill gas samples at solid waste landfills that receive construction and demolition (C&D) waste is usually much higher than at landfills that do not accept C&D. The higher concentrations of hydrogen sulfide are believed to be associated with the gypsum board component (e.g. wallboard) present in C&D material. The combination of gypsum, organic material, moisture and anaerobic conditions present in C&D landfills is believed to provide a favorable mixture and environment for bacteria to produce hydrogen sulfide gas. Concentrations of hydrogen sulfide detected in raw landfill gas samples, collected from within a landfill waste mass have ranged from 50,000 ppb to 15,000,000 ppb (50 ppm-15,000 ppm) for those landfills that accepted C&D solid waste. Landfills which do not accept C&D typically have much lower concentrations of hydrogen sulfide in the raw gas, usually less than 100,000 ppb (100 ppm). Hydrogen sulfide is generated as a result of a series of reactions that biologically reduce sulfate leached from gypsum contained in the C&D. In general, wallboard consists of the gypsum core (CaSO₄.2H₂O) with facing and backing consisting of paper. The microorganisms responsible for generating hydrogen sulfide include sulfate-reducing bacteria and sulfur-reducing bacteria. Sulfate reducing bacteria require the following to produce hydrogen sulfide: a sulfate source (gypsum), a carbon source (organic material), anaerobic conditions, and moisture.

Methane production also varies greatly from landfill to landfill depending on site-specific characteristics such as waste in place, waste composition, moisture content, landfill design and operating practices, and climate. Unless captured first by a gas recovery system, methane generated by the landfill is emitted when it migrates through the landfill cover. During this process, the soil oxidizes approximately ten percent of the methane generated, and the remaining 90 percent is emitted.

FIG. 2 shows a cycle of biogas production from landfills. There are four chemical stages of biogas production in landfills, hydrolysis, acidogenesis, acetogenesis, and methanogenesis. The first stage is hydrolysis. Landfill waste includes carbohydrates, fats and proteins. In the hydrolysis stage (1), carbohydrates are converted to sugars, fats are converted into fatty acids, and proteins are converted to amino acids.

The second chemical stage of biogas production is acidogenesis (2). IN this stage the sugars and fatty acids are converted into carbonic acids and alcohols. THe acidogenesis process also produces hydrogen, carbon dioxide, and ammonia.

The third stage of biogas production acetogenesis (3). In this stage the carbonic acids and alcohols are converted inot acetic acid, hydrogen, and carbon dioxide.

The fourth chemical stage of biogas production is methanogenesis (4). In this stage the acetic acid and hydrogen and converted into methane, and carbon dioxide. The methane can be captured into a landfill gas supply or a gas recovery system, and used to produce energy such as electrical power for a power grid.

### Sediments

A sequence of microbially-mediated oxidation and reduction reactions of decreasing thermodynamic energy yield occurs with depth in sediments containing moderate to large amounts of organic carbon. If sufficient organic matter is present after aerobic utilization of all the dissolved oxygen and establishment of anoxic conditions, these reactions result in the reduction of a number of dissolved species, including iron, manganese and sulfate, followed by generation of methane. Sulfate reduction and methanogenesis are considered to be mutually exclusive processes in sediments. Sediments containing organic matter is an example of a biogas generating medium. Typically, methanogenesis does not occur until all dissolved sulfate has been reduced to sulfide, resulting in the presence of a methanogenic zone beneath the sulfate-reducing zone. In effect, sulfate-reducing bacteria preferentially utilize a common organic carbon substrate until virtually all sulfate is depleted.

Hydrogen sulfide is an exceedingly important substance in the aquatic environment. Its presence and reactions rank with photosynthesis, algal respiration, and the iron cycle in the establishment of the electron (Eh) and proton (pH) activity of the aquatic environment. Hydrogen sulfide is an extremely active chemical and biochemical participant in these biogeochemical transformations.

Sedimentary production of hydrogen sulfide can increase the oxygen demand rate of sediment leading to a reduction in dissolved oxygen in overlying waters.

It has generally been accepted that the only significant route or pathway of hydrogen sulfide production in the aquatic environment is sulfate reduction, the microbial reduction of higher oxidation state inorganic sulfur, primarily sulfate, which serves as the terminal electron acceptor from the anaerobic oxidation of organic matter in the respiratory process. The principal sulfate reducing bacterium is Desulfovibrio desulfuricans. However, it is common knowledge that hydrogen sulfide is a product of the microbially-mediated anaerobic decomposition of the sulfur fraction of proteinaceous matter (putrefaction) and there is evidence that putrefaction can play a very significant role in hydrogen sulfide production in anoxic sediment under some conditions.

Methane is an endpoint of anaerobic organic carbon decomposition in oxygen-depleted sediments. Methane is produced in sediments by anaerobic methanogenic archaea. Their activity depends on many environmental factors, such as supply of organic matter, availability of electron acceptors and temperature. Although over 90% of the methane produced can be oxidized in the uppermost anaerobic sediment layers and water column, high methane emissions have been detected from lakes and reservoirs which contain large amounts of degradable organic matter in their sediments. FIG. 3 shows fluxes of methane and hydrogen sulfide from deep sediments. In this example, algae is growing in a natural environment, such as an ocean or lake. The algae absorb CO₂ while growing and during the life cycle. The CO₂ is asorbed from the air and from the water. O₂ is produced by the algae and released into the water. When the algae die, the organic matter from dead algae and other organic carbon is deposited onto and into the sediments of the natural environment, forming an environmental medium. Environmental mediums include but are not limited to sludge, soil, water, and gas. As the organic material is digested by in an anaerobid process, hydrogen sulfide and CO₂ is produced, especially when high concentrations of sulfate are present in the environmental medium. Methane is also produced, and seeps to through the environmental medium, with some of the methane escaping the environmental medium. Algae and other organic material in sediment is an example of a biogas generating medium.

### Sewer Systems

Hydrogen sulfide in sewer systems is a well-known problem due to odorous problems and corrosion on the walls of the sewer pipes. On the other hand the methane formation has not received as much attention. A few studies have been executed on rising mains since these are theoretically the ones with the highest potential for methane formation. Gravity mains are the most common kind of pipes but they are still poorly investigated. Investigations of these are necessary if methane formation in sewer systems shall be fairly evaluated. Factors that affect methane formation are among other things the presence of organic carbon in anaerobic conditions. The amount of methane formed per volume wastewater depends on the ratio between the inner surface wall area covered with biofilm and the volume of the pipe. This theory has been proved, not only in studies on rising mains but also correlate well with formation of hydrogen sulfide.

If methane formation is present and is somewhere in the sewer released to the atmosphere without being noticed, the carbon footprint of the sewer system is higher than previously thought. Besides the consideration of carbon footprint, methane emissions is also a risk since it is explosive in air at a certain concentration, thus dangerous environments could possibly occur.

Organic matter in a sewer system is an example of a biogas generating medium. For example, as shown in FIG. 4, a sewage typically contains biofilm on the sides of the pipe, where organic material is digested producing biogases including H₂S and CH₄.

### BRIEF DESCRIPTION OF THE DRAWINGS

FIG. 1 shows a diagram of cycle of manure uses, and methane production from manure.

FIG. 2 shows a cycle of biogas production from landfills.

FIG. 3 shows fluxes of methane and hydrogen sulfide from deep sediments.

FIG. 4 shows a sewage pipe and biofilm formation.

FIG. 5 shows the breakdown of organics into biogas.

FIG. 6 shows a graph of the methane concentrations listed in Table 3.

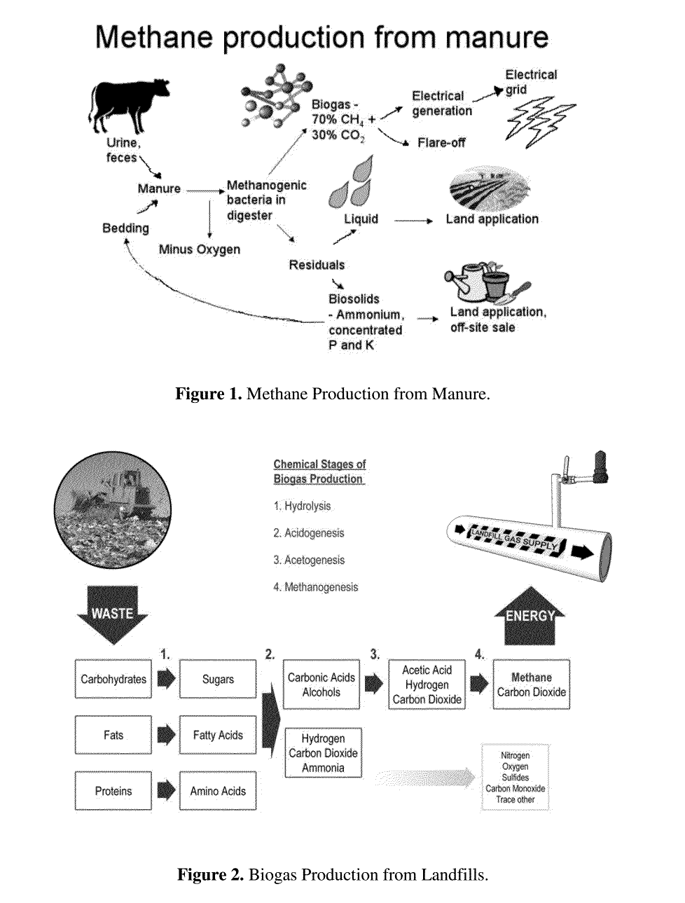

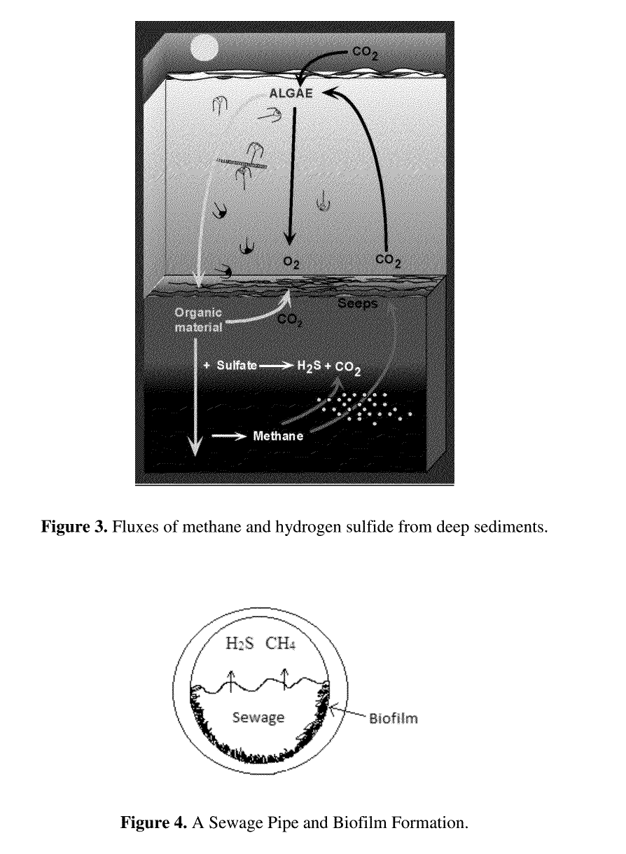

### SUMMARY OF THE DISCLOSURE

A method for inhibiting methane and hydrogen sulfide production from anaerobic digester systems with a biogas generating medium is disclosed. The biogas generating medium is contacted with an effective amount of a composition comprising red yeast rice and iron oxide to cause inhibition of methane and hydrogen sulfide production, and is useful in biogas generating medium from animal farms, including a swine, cattle or chicken farms. The method is useful to inhibit methane and hydrogen sulfide production in sewage systems, landfills, and sediment containing organic carbon. A disclosed inhibiting composition blocks 3-hydroxy-3-ethylglutaryl coenzyme A (HMG-CoA) reductase, and 8-hydroxy-5-deazaflavin (coenzyme F₄₂₀) in the methane production pathway, and includes lovastatin produced from red yeast rice, and iron oxide. A disclosed inhibiting composition prevents hydrogen sulfide formation with a competing reaction resulting into iron sulfate via reaction with iron (II) having been formed within the reducing environment from iron (III) oxide.

The disclosed methods provide techniques for inhibiting both the hydrogen sulfide and the methane production in anaerobic digester systems such as animal (swine, chicken, cattle) farms, landfills, sediments and enclosed sewer structures by the addition of an environmental friendly mixture of ingredients each one of which is targeting a different compound.

The mixture mainly consists of red iron oxide and red yeast rice extract. The iron oxide addresses the excess hydrogen sulfide production, by transforming it into insoluble iron sulfide, while the red yeast extract targets the methane production from methanogenic bacteria by depressing the action of various enzymes and coenzymes that play a key role in the methane production. An additional product that exclusively targets the methane production and consists of red yeast rice extract can also be introduced.

### DETAILED DESCRIPTION

Biogas is commonly produced by anaerobic digestion as part of the treatment of wet organic waste. This occurs in municipal wastewater and sewage treatment plants, industrial operations that have liquid wastes containing organic material, and on types of farms where animals are kept or held in a small area, such as pig or poultry farms.

Biogas is a mixture of mainly methane and carbon dioxide with very small amounts of hydrogen sulfide and other impurities. Table 1 below shows the concentration of hydrogen sulfide in biogas, as well as the concentrations of other major component gases, carbon dioxide and methane.

TABLE 1
Composition of Various Forms of Biogas

| Substrate | H₂S | CO₂ | CH₄ | |
|---|---|---|---|---|
| (by volume) | (ppm) | (%) | (%) | Source |
| Swine Waste | 600-4000 | 40 | 60 | (Pagilla, Kim and |
| | | | | Cheunbarn, 2000) |
| Cattle Manure | 600-7000 | 40 | 60 | (Bothi, 2007) |
| Landfill Wastes | 0-2000 | 30-50 | 50-70 | (Shin et al., 2002) |

Biogas from manure is generated biologically by anaerobic digestion of the complex organic molecules. Under anaerobic conditions microorganisms break down the organic debris until the carbon in the debris is in either its most reduced or its most oxidized state. Roughly sixty percent of the carbon is reduced and volatilizes as methane while the rest of the carbon volatilizes as carbon dioxide.

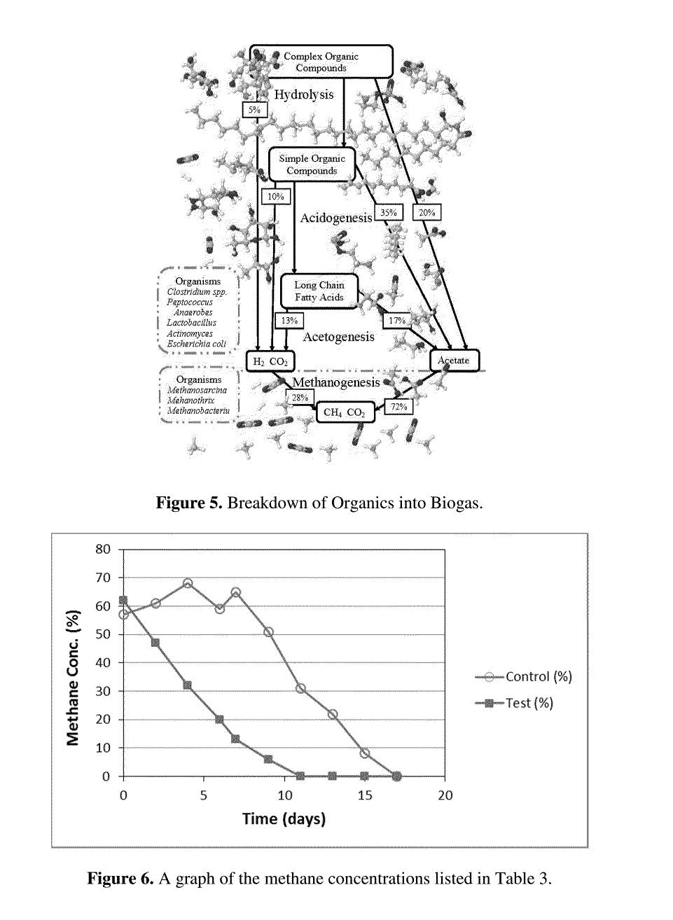

As many as 138 different microorganisms contribute to the production of biogas from swine manure with the majority of them being strict anaerobes. As presented in FIG. 5, these microorganisms can be broadly classified into two physiologically distinct groups. The first group breaks down the complex organics into simpler organic molecules (hydrolytic and fermentor organisms) and includes clostridium spp., peptococcus anaerobes, lactobacillus, actinomyces, and escherichia coli. The second group uses the simple organic molecules, particularly acetate and hydrogen, to make methane (methanogenic organisms) and includes methanosarcina, methanothrix, and methanobacteriu.

While manure is being digested, very small bursts of hydrogen sulfide will bubble out and accumulate as a small portion of biogas. Hydrogen sulfide is produced during hydrolysis when certain organisms break down the essential amino acid methionine. In the methanogenic stage hydrogen sulfide production continues because a different group of sulfate reducing organisms can use fatty acids, particularly acetate, as a substrate. As shown in an example of FIG. 5, complex organic material breaks down in the hydrolysis stage into simple organic compounds and produces H₂ and CO₂ and acetate. The simple organic compounds are broken down in the acidogenesis stage into long chain fatty acids and this stage also produces H₂ and CO₂ and acetate. The acetogenesis stage produces H₂ and CO₂ and acetate from the long chain fatty acids. The percentages shown in the drawing give approximate percentages of the total biogas produced from all three of the first three stages of the process shown in FIG. 5. For example in the hydrolysis stage approximately 5% of the total biogases in the first three stages are produced as H₂ and CO₂. In the methanogenesis stage, H₂ and CO₂ and acetate combine to create CH₄ and CO₂.

In an example embodiment iron oxide prevents hydrogen sulfide production by creating a competing reaction with the sulfide production. The competing reaction creates a reducing environment for hydrogen sulfide production and results in an iron sulfate through a reaction with iron (II) which is formed from iron (III) oxide in the reducing environment.

### Hydrogen Sulfide Treatment Process

The oldest commercial process for removing H₂S is iron sponge, which has been available for over 100 years. Iron sponge consists of hydrated iron oxide impregnated onto a carrier media like ceramic balls or redwood bark. A tank is then filled with the activated media forming a bed. As biogas passes through the bed a chemical reaction between the iron oxide and H₂S forms an iron sulfate, thus removing the H₂S from the biogas. A drain is located at the bottom of the vessel is used to remove water produced by the reaction. The activated media will eventually be spent and require replacement. The life of the media is a function of the H₂S concentration and the gas flow rate. The presence of oxygen in the gas will reactivate the iron oxide surface coating generating elemental sulfur in the process.

The sulfur removal capacities of iron oxide range from 0.20-0.72 kg of hydrogen sulfide for every one kg of iron oxide.

Fe₂O₃₍ₛ₎+3H₂S_{(g)}→Fe₂S₃₍ₛ₎+3H₂O₍ₗ₎ (1)

Apart from the reaction of iron oxide as shown above, several other reactions do occur during scrubbing of biogas with iron oxide, including:

Fe₃O₄₍ₛ₎+4H₂S_{(g)}→3FeS₍ₛ₎+4H₂O₍ₗ₎+S (2)

Fe₃O₄₍ₛ₎+6H₂S_{(g)}→3FeS₂₍ₛ₎+4H₂O₍ₗ₎+2H_{2(g)} (3)

FeS₍ₛ₎+S_{(g)}→FeS₂₍ₛ₎ (4)

In water systems, the reaction of dissolved hydrogen sulfide with iron hydroxides is occurring via a reductive-dissolution mechanism based on the following sequence of reactions, whereby sulfide initially forms a complex at the oxide surface, followed by electron transfer between the sulfide and Fe³⁺:

### Surface Complex Formation:

>FeOH+HS⁻→Fe^{III}S⁻+H₂S (5)

### Electron Transfer:

>Fe^{III}S⁻→>Fe^{II}S (6)

The S^{•-} Free Radical is then Released Followed by Fe²⁺ Dissolution:

>Fe^{II}S^{•-}+H₂O→>Fe^{II}OH₂⁺+S^{•-} (7)

>Fe^{II}OH₂⁺→new surface site+Fe²⁺ (8)

### Methane Treatment Process

Biological methane formation is a microbial process catalyzed by methanogens. The methanogenic pathways (or methane production pathways) of all species have in common the conversion of a methyl group to methane; however the origin of the methyl group varies. Most species are capable of reducing carbon dioxide (CO₂) to a methyl group with either a molecular hydrogen (H₂) or formate as the reductant. Methane production pathways in methanogens that utilize CO₂ and H₂, involve specific methanogen enzymes, which catalyze unique reactions using unique coenzymes.

The disclosed method demonstrates the effective use of naturally-occurring statins combined with the use of the hydrogen sulfide-inhibiting iron oxide to assess the methane produced from the enclosed anaerobic digester systems. The naturally-occurring statins, such as Lovastatin (C₂₄H₃₆O₅) can be obtained by the use of the commercially available red yeast rice extract. According to researchers, red yeast rice, which is an Asian dietary staple made by fermenting yeast (Monascus purpureus) on rice, contains active ingredients of the statin drugs such as Lovastatin. Methanogens are a diverse group of strict anaerobes which are widely distributed in nature and can be found in a variety of permanently anoxic habitats like flooded soils, sediments, sewage-sludge digesters or the digestive tract of certain animals. All known methanogens are affiliated to the Archaea and are extremely sensitive to oxygen. The hallmark feature of methanogens is the reduction of C-1 compounds (e.g., CO₂, methanol, formate, or N-methyl groups) to methane (CH₄). Some enzymes and cofactors are unique for this metabolic pathway and therefore only found in methanogens. Coenzyme F₄₂₀, and coenzyme A are two of the most important cofactors during the generation of methane.

Coenzyme F₄₂₀ or 8-hydroxy-5-deazaflavin, is a two electron transfer coenzyme that is involved in redox reactions in methanogens in many Actinobacteria, and sporadically in other bacterial lineages. It occurs at varying levels in all methanogenic species and has also been identified in Streptomyces griseus and Anacystis nidulans. One of the characteristics of F₄₂₀ is that it acts as an electron donor for two steps in the reduction of CO₂ to a methyl group. The F₄₂₀-dependent NADP oxidoreductase enzyme from Methanobrevibacter smithii catalyzes the important electron transfer step during methanogenesis between NADP⁺ and F₄₂₀. During the reaction, NADP is reduced to NADPH by accepting one or more hydrides (H⁻) from F₄₂₀. This is an important step of methane formation in methanogen bacteria such as M. smithii. Therefore, the NADP oxidoreductase enzyme plays a vital role in the formation of methane.

Sharma et al. (2011) determined a 3D model structure of the F₄₂₀-dependent NADP oxidoreductase enzyme from M. smithii. Based on their protein model, they detected that these residues are making a ligand binding site pocket, and they found that ligand F₄₂₀ binds at the protein cavity. The inhibitor compounds lovastatin and compactin (mevastatin) show more affinity for the model protein as compare to the natural ligand F₄₂₀. They share the same cavity as by F₄₂₀ and surround by similar residues. Therefore, the inhibitor compounds lovastatin and compactin (mevastatin) were very effective in blocking the activity site for methane production since the enzyme was unable to bind with the substrate, resulting in decreased methane production.

The acetyl coenzyme A (CoA) pathway commonly referred to as the Wood-Ljungdahl pathway or the reductive acetyl-CoA pathway is one of the major metabolic pathways utilized by methanogenic bacteria. This specific pathway is characterized by the use of hydrogen as an electron donor and carbon dioxide as an electron acceptor to produce acetyl-CoA as the final product. The acetyl-CoA pathway begins with the reduction of a carbon dioxide to carbon monoxide. The other carbon dioxide is reduced to a carbonyl group. The two major enzymes involved in these processes are carbon monoxide dehydrogenase and acetyl CoA synthase complex. The carbon dioxide that is reduced to a carbonyl group, via the carbon monoxide dehydrogenase, is combined with the methyl group to form acetyl-CoA. The acetyl-CoA synthase complex is responsible for this reaction. The 3-hydroxy-3-methylglutaryl coenzyme A (HMG-CoA) reductase, is the critical enzyme in methane production in Methanobrevibactor strains, since Archaea are the only bacteria known to possess biosynthetic HMG-CoA reductase.

Lovastatin is a secondary product of idiophase (secondary phase) of growth of fungi and is an inhibitor of enzyme 3-hydroxy-3-ethylglutaryl coenzyme A (HMG-CoA) reductase, a key enzyme also in cholesterol production pathway in humans. There is a similarity between cholesterol formation in human and cell membrane formation in the Archaea (methanogens) as the lipid side of phospholipids in the cell membrane of Archaea is isoprenoid chains. Isoprenoid formation is an intermediate step of cholesterol production pathway (Mevalonate pathway) and HMG-CoA reductase is also a key enzyme for its production. Therefore, as an inhibitor of HMG-CoA reductase, lovastatin suppresses isoprenoid production and thus cholesterol synthesis and methane formation in the Archaea.

Wolin and Miller (2005) showed that lovastatin significantly reduced growth and activity of pure methanogenic bacteria without any negative effect on cellulolytic bacteria. Further studies have shown that red yeast rice can successfully inhibit the key enzyme HMG-CoA reductase, resulting in the inhibition of methanogenic activity. Miller and Wolin (2001) also used Lovastatin to inhibit the formation of the key precursor mevalonate. Mevalonate is formed by reduction of HMG-CoA. Based on their results they found that lovastatin inhibited the growth of Methanobrevibacter and subsequently the methane production. In fact 4 nmol/ml of culture medium resulted in 50% inhibition of growth and concentrations 10 nmol/ml of culture medium completely inhibited growth. Methane formation was also significantly inhibited. At the same time the populations of the non-methanogens were not affected.

### Examples

Two bench scale studies were performed to test the effectiveness of the methane inhibitor red yeast rice (RYR).

### Purpose

The purpose of the two laboratory studies was to evaluate the effectiveness of Methane Inhibitor Red Yeast Rice (MIRYR), a composition developed by the inventors herein. The product was designed to inhibit methane production in environments where methanogens are established and active.

### Materials and Methods

### Laboratory Study 1

Two anaerobic reactors were utilized, a Control and a Test reactor. The two reactors were seeded with biomass treating expired dietary supplement, which contained an active methanogenic population. The reactors were fed once per week, and were operated as anaerobic sequencing batch reactors.

During the first week of startup, the reactors contained only the methanogenic culture, without soil. After one week, silty sand was added, resulting in a slurry having a solids concentration of 20% by weight. The reactors were operated for another week with the silty sand, to ensure that the sand did not affect methanogenic activity. The bioreactors were 2.5 L in volume, containing 2 L of slurry. The reactors were airtight and were especially designed for anaerobic reactions. The reactors were maintained at laboratory temperature 22° C.-24° C. The reactors were operated by feeding with dietary supplement once a week. The target initial chemical oxidation demand (“COD”) concentration after feeding was 2000 mg/L. Throughout the week, the volume of biogas produced was measured as follows. A syringe was inserted periodically into a septum-filled port in the top of the reactor to collect a gas sample for methane content. The methane content of the biogas samples was then quantified by injecting into a gas chromatograph with a flame ionization detector (GC-FID). The reactors had dedicated probes to measure pH and oxidation reduction potential (“ORP”). After each cycle (i.e., before feeding), a probe was inserted into the reactor to measure total dissolved solids (“TDS”), and a sample was collected to measure COD. The mixer was turned off during sampling and feeding to minimize the introduction of oxygen into the reactor contents.

The Test reactor was initially dosed with a 40 g/L concentration of Methane Inhibitor RYR (MIRYR). One week later the Control was dosed with 20 mg/L MIRYR.

### Laboratory Study 2

Two test aliquots were prepared under a nitrogen atmosphere in a glove box as follows:

1. A 240 mL amber glass screw-cap septum bottle was filled with 100 g of dry soil (˜70 mL).
2. Deoxygenated deionized water was slowly added to the soil to saturate the soil; an additional 40 mL of water was then added to the soil.
3. Manure slurry was added to yield a 1 weight percent manure dose to the soil.

Once the bottle was sealed it was removed from the glove box. The soil was kept in the dark (by wrapping with foil) at room temperature (˜22° C.). A needle connected to a polyethylene tube was pushed through the bottle septum and the tube outlet was placed in an inverted graduated cylinder in a water bath. The gas generation rate was recorded as the water was displaced over a period of 10 days.

The methane reduction trial included two sample formulations, with and without MIRYR, for a total of 4 samples. The bottles were sampled 0.5, 1.5, 5, 12, and 19 days following the sample preparation.

### Results

### Laboratory Study 1

The first two weeks of the studies were the Startup Period, and the second two weeks were the Test Period. The Startup Period established the methanogenic population in the two reactors. During the first week of startup, the reactors were operated without the silty sand, and the second week they were operated with the silty sand (20% by weight). The Test Period started with the dosing of the Test reactor with MIRYR (40 g/L). During the first week of the Test Period the Control was maintained as a proper control, with no MIRYR added. Because the 40 mg/L dose of MIRYR reduced methane production in the Test reactor, it was decided to dose the Control reactor with 20 g/L of MIRYR during the second week of the Test Period. The Test Period lasted 17 days. Table 2 lists the volume of biogas production, pH values, and the concentrations of COD, ORP, and TDS measured in the Control and Test reactors during the studies. The volume of biogas produced each feed cycle (i.e., each week) in the reactors ranged between 72-82 mL. It is notable that the volume of gas was not affected by the introduction of silty sand during week 2 of the Startup period. The addition of 40 mg/L of MIRYR to the test in the first week of the Test period and the addition of 20 mg/L of MIRYR during the second week of the Test period did not appreciably impact biogas volume in the reactors. The COD measurements after each sequencing batch reactor cycle ranged from 56 to 108 mg/L. The reactors were fed 2000 mg/L each cycle, so the COD concentrations in Table 2 demonstrate that the COD was consumed by

the anaerobic culture. Values of pH ranged between 6.1 and 6.4. Values of ORP were all close to −300 mV, which is typical of methanogenic conditions. The TDS in the reactors ranged from approximately 1200 to 1250 mg/L.

TABLE 2
A list of the biogas volume, pH values, and concentrations
of COD, ORP, and TDS in the Control and the Test
reactors throughout the studies.

| | | | | | |
|---|---|---|---|---|---|
| | Gas Vol. | COD | | ORP | TDS |
| Period | (mL) | (mg/L) | pH | (mV) | (mg/L) |

CONTROL

| | | | | | |
|---|---|---|---|---|---|
| Startup-Week 1 | 81 | 56 | 6.4 | −302 | 1213 |
| Startup-Week 2 | 72 | 91 | 6.3 | −306 | 1241 |
| Test-Week 1 | 75 | 61 | 6.2 | −289 | 1258 |
| Test-Week 2 | 73 | 108 | 6.3 | −296 | 1220 |

TEST

| | | | | | |
|---|---|---|---|---|---|
| Startup-Week 1 | 79 | 72 | 6.2 | −285 | 1244 |
| Startup-Week 2 | 75 | 83 | 6.2 | −298 | 1265 |
| Test-Week 1 | 82 | 62 | 6.1 | −306 | 1263 |
| Test-Week 2 | 72 | 97 | 6.4 | −287 | 1247 |

Table 3 lists the methane content measured in the biogas generated in the reactors during the 17-day study period. FIG. 6 shows a graph of the methane concentrations listed in Table 3. During the Startup Period, methane concentrations varied from approximately 55% to 70%, which indicates an active methanogenic culture. The MIRYR dose of 40 mg/L in the Test reactor reduced the methane content of biogas from 62% to below detection (0.05%) after 11 days. The methane concentration remained below detect in the Test reactor until day 17, when the reactors were dismantled. The MIRYR dose of 20 mg/L in the Control reactor on day 7 reduced the methane content of biogas from 65% to below detection (0.05%) by day 17 (i.e., after 10 days). During the Test period, the volume of biogas produced in the Test and Control reactors did not change appreciably (Table 2), only the methane concentration of the biogas was changed.

TABLE 3
A list of the methane concentrations (%) measured in the biogas during
the Test Period (i.e., after dosing with methane inhibitor).

| Activity | Time (days) | Control (%) | Test (%) |
|---|---|---|---|
| dosed Test (40 mg/L) | 0 | 57 | 62 |
| | 2 | 61 | 47 |
| | 4 | 68 | 32 |
| | 6 | 59 | 20 |
| dosed Control (20 mg/L) | 7 | 65 | 13 |
| | 9 | 51 | 6 |
| | 11 | 31 | 0 |
| | 13 | 22 | 0 |
| | 15 | 8 | 0 |
| | 17 | 0 | 0 |

It is understood that the invention is not limited to the disclosed embodiments and examples, but is intended to cover various modifications and equivalent arrangements included within the spirit and scope of the appended claims.

### Laboratory Study 2

Table 4 lists the methane content measured in the biogas generated in the reactors during the 19-day study period. The first soil formulation (SF1) that contains 20% of the MIRYR (approximately 40 mg/L in solution) showed great effectiveness in inhibiting the methane production by 96% during the 19-day sampling interval. Similarly at the same time fragment the second soil formulation (SF2) resulted into a 25% decrease in methane production.

TABLE 4
A list of the methane concentrations (%) measured in the biogas
of the seven sample formulations during the Test Period.

| | SF1 (no | SF1 (with 20% | SF2 (no | SF2 (with 10% |
|---|---|---|---|---|
| Time (days) | MIRYR) | MIRYR) | MIRYR) | MIRYR) |
| 0.5 | 1.0 | 0.0 | 1.0 | 0.0 |
| 1.5 | 1.0 | 2.0 | 7.0 | 8.0 |
| 5 | 5.0 | 5.0 | 0.0 | 5.0 |
| 12 | 1.39 | 0.79 | 0.94 | 0.86 |
| 19 | 3,217 | 140 | 2,685 | 2,023 |

SF: Sample Formulation

Once again, it is understood that the invention is not limited to the disclosed embodiments and examples, but is intended to cover various modifications and equivalent arrangements included within the spirit and scope of the appended claims.

What is claimed is:

1. A method for inhibiting methane and hydrogen sulfide production from anaerobic digester systems, such as animal farms, and other biogas generating media, including landfills, sediments and sewer systems; the method comprising contacting the biogas generating medium with an effective amount of a composition comprising red yeast rice and iron oxide to cause inhibition of methane and hydrogen sulfide production.

2. The method of claim 1 wherein the methane and hydrogen sulfide production is being released in an environmental medium.

3. The method of claim 2 wherein the environmental medium is sludge, soil, water or gas.

4. The method of claim 1 wherein the source biogas generating medium is from animal farm waste.

5. The method of claim 4 wherein the animal farm waste is from a swine, cattle or chicken farm.

6. The method of claim 1 wherein the source biogas generating medium is waste from sewage systems.

7. The method of claim 1 wherein the source biogas generating medium is waste in landfills.

8. The method of claim 1 wherein the source biogas generating medium is sediment containing organic carbon.

9. The method of claim 1 wherein the biogas generating medium has a methane production pathway and a hydrogen sulfide production pathway.

10. The method of claim 1 wherein said inhibiting composition comprising red yeast rice targets the methane production pathway and iron oxide targets the hydrogen sulfide production pathway.

11. The method of claim 1 wherein said composition blocks the 3-hydroxy-3-ethylglutaryl coenzyme A (HMG-CoA) reductase in the methane production pathway.

12. The method of claim 1 wherein said composition blocks the 8-hydroxy-5-deazaflavin (coenzyme F₄₂₀) in the methane production pathway.

13. The method of claim 1 wherein said composition includes a naturally-occurring statin.

14. The method of claim 12 wherein the naturally-occurring statin is lovastatin.

15. The method of claim 14 wherein the source of lovastatin is red yeast rice.

16. The method of claim 1 wherein said anaerobic digester systems include a reducing environment, wherein said inhibiting composition prevents hydrogen sulfide formation with a competing reaction in the reducing environment and resulting into iron sulfate via reaction with iron (II) having been formed within the reducing environment from iron (III) oxide.

17. The method of claim 1 wherein the composition is a mixture of red yeast rice extract and iron oxide.

18. The method of claim 17 wherein the red yeast rice extract to iron oxide ratio of the composition is a first ratio for a first purpose, and is a second ration different from the first ratio, for a second purpose.
